(19) United States
(12) Patent Application Publication
(10) Pub. No.: US 2025/0347648 A1
(43) Pub. Date: Nov. 13, 2025

(54) ELECTROCHEMICAL ENERGY DIAGNOSTICS DEVICE FOR SAMPLE ANALYSIS

(71) Applicants: John William DILLEEN, Clackmannshire (GB); ENERGY DIAGNOSTICS LIMITED, Stirling (GB)

(72) Inventor: John William DILLEEN, Clackmannshire (GB)

(21) Appl. No.: 18/867,993

(22) PCT Filed: May 25, 2023
(86) PCT No.: PCT/GB2023/051387

§ 371 Date: Nov. 21, 2024

(30) Foreign Application Priority Data
May 25, 2022 (GB) 2207707.7

(51) Int. Cl. G01N 27/327; G01N 33/543

(52) U.S. Cl. CPC G01N 27/3277; G01N 27/3276; G01N 33/54333; G01N 33/5438

(57) ABSTRACT

Method and device for performing an electrochemical assays. A microfluidic device including anode and cathode electrodes capable of forming a Galvanic cell is described. In the presence of target analyte the Galvanic cell is completed and voltage or current can be measured and related to the amount of analyte present.

### FIELD OF THE INVENTION

This invention relates generally to fields of transduction analyte detection, biosensors and immunoassays.

More particularly, it relates to an electrochemical transduction device and associated detection method in which an analyte in a liquid sample or a substance to be assayed is measured using an electrical system comprising redox active particles, electrolytes and electrodes, capable of spontaneously forming a measurable Galvanic cell.

### BACKGROUND OF THE INVENTION

Immunoassay detection methods provide quantitative, semi-quantitative or qualitative detection of analytes of biological interest and they are widely use in multiple bioanalytical settings, such as biopharmaceutical analysis, food testing, clinical diagnostics, environmental monitoring, and the like.

Immunoassay detection methods may employ transduction devices to detect target analytes, such as optical transducers, electrochemical transducers, piezoelectric transducers, magnetic transducers, Surface Plasmon Resonance (SPR) Transducers, thermal transducers, or Radioimmunoassay (RIA) Transducers.

Optical transducers utilize light-based detection methods. Some common optical techniques used in immunoassay detection include: Fluorescence: Fluorescent labels attached to antibodies or antigens emit light when excited by specific wavelengths. Chemiluminescence: Chemiluminescent labels produce light as a result of a chemical reaction. Absorbance and Reflectance: These techniques measure changes in light absorbance or reflectance caused by the binding of the analyte-antibody complex.

Electrochemical transducers measure electrical signals generated by redox reactions. Some electrochemical techniques used in immunoassay detection include, but are not limited to, amperometry, potentiometry, and impedance spectroscopy.

In amperometry, the electric current resulting from the oxidation or reduction of an electroactive species produced during the immunoassay reaction is measured. The current is directly proportional to the concentration of the analyte. In potentiometry, potential changes resulting from redox reactions are measured. Ion-selective electrodes or pH electrodes are commonly used to measure the potential changes associated with the immunoassay reaction. Impedance-based techniques measure the electrical impedance changes caused by the binding events on the electrode surface. These changes can be correlated with the concentration of the analyte.

Piezoelectric transducers utilize the piezoelectric effect, where mechanical stress generates an electrical signal.

Magnetic transducers involve the use of magnetic particles conjugated with detection antibodies or antigens. The particles are detected and quantified using magnetic sensors or magnetoresistive sensors.

Surface Plasmon Resonance (SPR) Transducers detect changes in the refractive index of a thin metal film on a sensor surface.

Thermal transducers measure changes in temperature resulting from the binding of analyte-antibody complexes.

Radioimmunoassay (RIA) Transducers utilize radioactive isotopes as the detection method. It involves the use of a radioactively labelled antigen or antibody to measure the concentration of a specific analyte in a sample.

Electrochemical detection is widely deployed in analytical chemistry applications, and particularly in medical diagnostics. Simple enzyme based biosensors for measuring glucose in blood in the home, and more complex immunoassay systems for measuring biomarkers in blood in point of care settings form the background of this invention.

Detection of glucose in blood can be performed using handheld electronic readers with biosensor test strips comprising electrodes and enzyme reagent chemistries in capillary fill chambers. Immunoassays for cardiac biomarkers of heart disease can be performed in portable lab instruments, comprising electronics readers with electromechanics and test cartridges with antibody reagents, detection labels and microfluidic sample channels and chambers. Devices such as these are already widely commercially available.

Particle based immunoassays are described in U.S. Pat. No. 10,725,032 B2, and references therein, which are incorporated herein by reference. In such immunoassays, the capture and location of sample analytes can be controlled by immobilising antibodies specific to the analyte to stationary surfaces and mobile particles, and by mechanically controlling the microfluidics and movement of the sample and assay reagents and particles. Another method of control is by employing magnetically susceptible particles which can be attracted to surfaces with static or movable magnets. The magnetic particles can be coated to specifically bind to analytes and other assay reagents and particles. The most common particle based systems are found in home and laboratory test lateral flow assays with direct visual or instrument aided optical detection. Particle based assays with electrochemical detection are employed in certain commercial portable point of care instruments and laboratory based analysers.

Electrochemical detection systems may comprise enzyme functionalised detection particles that control the position of the analyte to enzymes and reagent chemistries close to a detection electrode. Detection particles themselves can be comprised of redox active chemistries which can be captured and detected at electrodes to indicate the presence and amount of the analyte. The particles can be a metal or metal oxides which can react at the detection electrode in the presence of reactive chemistries or by applying a voltage and current at the electrode to react and measure the metal particle amounts. Systems employing magnets and magnetically susceptible particles can be used to control the position of the electrochemically active component relative to the detection electrode. Magnetically assisted binding assays utilising a magnetically responsive reagent are well known in the art, e.g., U.S. Pat. Nos. 6,294,342, 7,045,364, both of which are hereby incorporated by reference.

Common electroanalytical methods for detection are amperometry, voltammetry, potentiometry and coulometry. A lesser used method is galvanometry. U.S. Pat. No. 10,598,625, the content of which is hereby incorporated by reference, describes a method of employing particles formed from a first metal conjugated to analytes. The analyte conjugated to the particle formed from the first metal can be accumulated at a working electrode. The first metal can be galvanically exchanged with ions of a second metal to form a layer of the first metal at the working electrode. The first metal can then be electrochemically detected and/or quantified by applying a voltage or current. However, the detection reaction is not spontaneous and a voltage current must be applied for the galvanic exchange, which can also cause other non-specific species to react and interfere with the detection method. A similar approach is described in WO 2016/025539A1, the content of which is hereby incorporated by reference.

Within the context of this disclosure, the terms “Voltaic” or “Galvanic” are not a specific method in the context of electrochemical transducers or immunoassay detection. The term “voltaic” typically refers to a voltaic cell or a galvanic cell, which is an electrochemical cell that produces an electric current from a spontaneous chemical reaction. Voltaic cells are commonly used as power sources, such as batteries.

In the realm of biosensors or immunoassay detection using electrochemical techniques, existing specific methods include amperometry, potentiometry, impedance spectroscopy, cyclic voltammetry, square wave voltammetry, and differential pulse voltammetry. These methods utilise electrochemical principles to detect and quantify analytes based on the electrical signals generated by redox reactions or changes in impedance. However, most of these techniques require the application of electrical current to the detection system, and this current can activate interferents present in said system and increase background signals, therefore reducing the sensitivity of the detection method.

An electrochemical amplification immunoassay using bi-electrode signal transduction system is presented in Talanta, Volume 71, Issue 5, 30 Mar. 2007, Pages 2029-2033, the content of which is hereby incorporated by reference. The system employs a split cell with salt-bridge, and uses two separate electrodes, an ‘immunoelectrode’ and a detection electrode to form a galvanic cell to implement the redox reactions on the two different electrodes. The enzyme-generated reductant in the anode region is electrochemically oxidized by an oxidant (silver ions) in the cathode apartment. The accumulated silver is measured using voltammetry, which is susceptible to interference from other redox active impurities or naturally occurring substances in the sample. Also, an enzyme-catalysed silver deposition on irregular-shaped gold nanoparticles for electrochemical immunoassay of alpha-fetoprotein is presented in Analytica Chimica Acta, Volume 755, 28 Nov. 2012, Pages 62-68, the content of which is incorporated hereby by reference. The enzymatically catalytic deposition of silver on the electrode is measured by voltammetry, which is susceptible to interference. For example, in glucose biosensors uric and ascorbic acid in blood can interfere electrochemically at the electrode giving false readings.

Employing a galvanic couple as a power source for iontophoretic drug delivery devices is well known in the art, e.g., U.S. Pat. No. 6,653,014, the content of which is incorporated hereby by reference. These inventions however do not relate to assay devices.

Other particle based immunoassay's incorporating metal or metal oxide particles and optical detection are commonly used. Gold and silver nano particles are employed in lateral flow optical detection systems, e.g. U.S. Patent No., 2021/0156856 A1, the content of which is hereby incorporated by reference. U.S. Pat. No. 8,383,337, which is hereby incorporated by reference describes a method which involves binding a probe to an analyte present in a sample. The probe consists essentially of a binder bonded to a metal particle that is capable of releasing metal ions when contacted with a reagent solution. On reacting, metal ions are released from the metal particle and an optical signal observed indicating the presence or amount of the analyte in the sample. The metal particle consists essentially of a metal oxide such as functionalised zinc oxide, which can be functionalised by an APTES biotinylation process and employed with standard magnetic streptavidin bead capture.

There is a need for an improved detection device and method which mitigates at least some of the issues experienced with existing electrochemical detection systems.

An electrochemical battery cell is a closed electrochemical system that converts chemical energy from spontaneous oxidation and reduction reactions directly into electric energy. A system where two electrodes of differing standard reduction half-cell potentials placed in a suitable electrolyte will spontaneously flow current when the electrodes are connected. This invention takes this fundamental chemical energetics into analytical applications which may be termed ‘energy diagnostics’.

### SUMMARY OF THE INVENTION

In a first aspect there is provided a galvanic detection device comprising at least one pair of detection electrodes, wherein at least one of:
a) At least one detection electrode comprises a capturing surface capable of capturing an analyte and/or particle; and/or
b) The galvanic detection device comprises a capturing system configured to travel to at least one detection electrode, the capturing system having a capturing surface capable of capturing an analyte and/or particle.

The galvanic detection device may comprise a capturing surface or a capturing system. The capturing surface or system may be configured to capture or bond to an analyte or particle for determination of the presence and/or quantity of that analyte in a sample. The capturing surface or system may be configured to capture or bond to an analyte or particle by any suitable means, for example by immunoassay, size exclusion, magnetic forces, chemical reaction, and the like. The capturing surface or system may be present on a functionalised transporter, electrical conductor and/or on a functionalised electrode. The capturing surface may be present on a particle, for example a magnetic particle. A capturing substance of the capturing surface or system may be an antibody or a protein. The capturing surface or capturing system may comprise a size exclusion filter. The size exclusion filter may be disposed on an electrical conductor and/or a detection electrode. The capturing surface or capturing system may be a capturing particle, such as a magnetic particle.

The galvanic detection device may comprise a detection particle and a capturing surface for capturing the detection particle. The detection particle may be configured to capture or bond to an analyte. The capturing surface may be configured to capture the detection particle. The detection particle and the capturing surface may be configured to form a sandwich complex with an analyte (in use). The detection particle may be functionalised with a biological moiety, such as an antibody or protein, for capturing an analyte. The detection particle may comprise a metal, such as the same metal as the cathode or anode detection electrode.

The capturing surface may comprise a biological moiety for capturing the detection particle. The capturing surface and the detection particle may each comprise a biological moiety which is complementary to the biological moiety of the other one of capturing surface or detection particle. For example, one of the capturing surface or the detection particle may comprise an antibody and the other of the capturing surface or the detection particle may comprise the antigen to said antibody.

The detection particle may comprise a capturing surface. For example, the detection particle may be functionalised so as to be able to form a complex with an analyte. The detection particle may comprise streptavidin or biotin. The detection particle may comprise an antibody. The detection particle may comprise a metal particle (e.g. zinc) functionalised with biotin or streptavidin and further functionalised with biological moiety (e.g. antibody). In preferred embodiments, the detection particle is a zinc particle functionalised with biotin and further functionalised with a biological moiety for binding to an analyte.

The capturing surface or system may be disposed on the detection electrode or electrodes. Alternatively, the capturing surface or system may be disposed on an element (e.g. particle) configured to travel to the detection electrode or electrodes (e.g. under a magnetic field), or by fluid pressure. The capturing surface or system may not be disposed on or not be configured to travel to the opposite electrode. An analyte may be configured to be captured directly at an electrode. Alternatively, an analyte may be configured to be captured at the electrode indirectly, by first forming a complex with a capturing system (e.g. (carrier) particle), and then the complex being configured to be captured at the electrode. The capturing surface or system (e.g. a particle), may be configured to react with an analyte at a detection electrode by chemical reaction or an electrochemical reaction.

The galvanic detection device may comprise a capturing surface for capturing the detection particle. The capturing surface may comprise streptavidin or biotin. In preferred embodiments, the capturing surface may comprise streptavidin for capturing detection particles, or complexes comprising biotin. The capturing surface (e.g. streptavidin) may be present on the surface of a detection electrode, or it may be present on the surface of a magnetic particle.

The capturing surface or system may be configured to form a complex with a target analyte. The complex may comprise or consist of a substance or analyte, an antibody to the substance or analyte, and a redox active material. The redox active material may be any suitable material that can form a Galvanic cell. The redox active material in the complex may be configured to allow electron transfer to an electrode. For example, the redox active material may be disposed close enough to a detection electrode in order to allow electron transfer.

In use, the galvanic detection device is configured to form an assay complex sandwich construct comprising a detection particle comprising a redox active material bound to a secondary antibody, an analyte from a sample, a primary antibody bound to biotin and streptavidin bound to a capturing surface.

The capturing surface or system (or complex) may be stationary. For example, the capturing surface or system (or the complex) may be bound to an element of the galvanic detection device (e.g. a detection electrode, a substrate, or the like. In use, the capturing surface or system may be configured to capture, react with, or bond to an analyte at a specified location to form a complex. Alternatively, the capturing surface or system may be mobile. For example, the capturing surface or system may be present in a substrate or electrolyte and may be capable of moving along the galvanic detection device towards a detection electrode. For example, the capturing surface or system may be configured to capture or bond to an analyte (in use) to form a complex, and the complex may be configured to travel or be transferred to a detection electrode to be quantified. The complex may be transferred by means of magnetism. Alternatively, the complex may be transferred by fluidic control and flow. The capture or bonding of an analyte with the capturing surface or system may modify the properties of the capturing surface or system so that the change can be detected electrochemically by at least one pair of detection electrodes. In other words, the properties of the complex may differ from the properties of the capturing surface or system. The change in properties when the complex is formed may be measured by the detection electrodes and this can be correlated to the amount of analyte present in a sample.

The device may comprise a control (or reference) electrode. There may be a control (reference) electrode for each pair of detection electrodes (anode/cathode pair). The/each control (or reference) electrode may have an established electrode potential. The/each control electrode may be configured measure background voltage or unknown interferent voltage. The reference electrode may be configured to be measured for quality control. The reference electrode may not comprise a magnet.

The control electrode may comprise an unfunctionalised surface. The pair of control electrodes may be a calomel electrode or a silver-silver chloride electrode (Ag|AgCl).

The galvanic detection device may comprise a single pair of detection electrodes. The galvanic detection device may comprise multiple pairs of detection electrodes. Each pair of detection electrodes may comprise a cathode and an anode. Each pair of detection electrodes may comprise a control electrode. In some embodiments, the galvanic detection device may comprise two, three, four, five, or six pairs of detection electrodes. In some embodiments, the galvanic detection device comprises two pairs of detection electrodes, each pair of detection electrodes comprising a control electrode.

At least one of the detection electrodes of the/each pair of detection electrodes may define an inactive form and an active form. The inactive detection electrode may be configured to be activated during an assay. For example, an inactive detection electrode may comprise a non-reactive conductor electrode. The non-reactive conductor electrode may be selected from a carbon electrode or a noble metal electrode (e.g., a gold electrode, or a platinum electrode). The inactive detection electrode/electrodes may be configured to receive a metal or metal salt during an assay and become active. The metal or metal salt may be selected from zinc, a zinc salt, silver, a silver salt, such as Ag/AgCl. In use, when the inactive detection electrode becomes active, the Galvanic cell may be completed.

In use, before an assay, the galvanic detection device may comprise at least one inactive conductive electrode or a pair of inactive conductive electrodes (e.g. a carbon electrode(s)), and electroactive materials (e.g. an electrolyte comprising a metal or metal salt). For example, the galvanic detection device may comprise carbon electrodes and an electrolyte comprising zinc particles and Ag/AgCl. During an assay, the zinc particles may be configured to travel to and deposit on the anode and the Ag/AgCl may be configured to travel to and deposit on the cathode. Therefore, the pair of detection electrodes may initially be inactive and become active anode and cathode electrodes as an assay progress, or one detection electrode may initially be active and the second one become active as an assay progresses.

The inactive detection electrode may be configured to become active by deposition of electroactive material on the surface of the electrode by any suitable means. For example, an electroactive material may be configured to be attracted to or bound to the surface of the detection electrode, for example if the electroactive material is coupled to a substance or particle which may be compatible with a capturing surface of the electrode, or which may be magnetically attracted to the surface of the electrode under the influence of a magnetic field generated by a magnet disposed adjacent to the detection electrode.

The anode and cathode may comprise different chemical agents. For example, the anode and cathode may comprise different metal salts. The metals salts from each pair of detection electrodes may be separated from other pairs of detection electrodes by a salt bridge or a temporary ion bridge. The temporary ion bridge may last the duration of detection and measurements. The salt bridge or ion bridge may connect the oxidation and reduction half-cells of the galvanic detection device. Without wishing to be bound by theory, the salt bridge may maintain electrical neutrality within the internal circuit. If no salt bridge were present, the solution in one-half cell would accumulate a negative charge and the solution in the other half cell would accumulate a positive charge as the reaction proceeded, quickly preventing further reaction, and hence the production of electricity. The salt bridge may be a channel containing electrolyte between the anode and cathode of each pair of detection electrodes. The channel may contain a common electrolyte for the cathode and the anode. In some embodiments, the cathode and the anode comprise different metal salts and salts stay separated. Therefore electrolyte in the channel between electrodes may be the salt bridge if we incorporate different metal salts to the anode and cathode which stay separated in the channel during the measurement.

The electrode may be flat and smooth where the particle size combination permits an active anode or cathode material to contact the electrode. The electrode can be porous. In a porous electrode, capturing particles are configured to be pulled into micro domains of the electrode, providing greater surface area and opportunity for the redox active material to touch the electrode.

The cathode electrode may comprise silver. The cathode electrode may comprise a silver and silver chloride blend (Ag|AgCI). The silver and/or silver chloride blend may be deposited on the surface of the cathode. The cathode electrode may comprise screen printed silver with silver chloride film. The cathode electrode may comprise a screen printed carbon film.

The anode may comprise zinc. The anode may comprise a zinc nano or micro particle. The zinc, zinc nanoparticle or zinc microparticle may be deposited on the surface of the anode. The anode electrode may comprise a screen printed carbon film.

At least one of the electrodes from the pair of detection electrodes (i.e. at least one of the detection cathode and/or the detection anode) may comprise a functionalised surface. The surface of the detection electrode may be functionalised in any suitable manner. For example, the surface may be functionalised so as to be capable of capturing galvanic redox material, in use. The surface of the detection electrode may comprise a capturing agent directly coupled to the surface, or a detection particle modified with the capturing agent. The capturing agent may comprise an enzyme, an antibody, a protein, a nucleotide, a redox active material (such as a metal or metal oxide), a magnetically susceptible material and the like. In some embodiments, the capturing agent may comprise biotin-streptavidin capturing system. In other embodiments, the capturing agent may comprise a bio recognition and/or a chemical recognition agent. In some embodiments, the capturing agent may comprise an aptamer. The surface of the detection electrode may be functionalised with the capturing agent. For example, the surface of the detection electrode may be functionalised with a capturing system comprising a size exclusion filter with electrical conductors and electrodes forming a measurable Galvanic circuit.

The capturing system may comprise a particle, such as a magnetically susceptible particle configured to be transferred to a detection electrode by a magnetic field or magnet behind the electrode, or be susceptible to other self-assembly means. The capturing system (or surface thereof) may be functionalised with a capturing agent. The analyte capturing agent may be an immunoassay, for example a sandwich type immunoassay where the analyte is configured to be held between the capture surface and the substance complex by antibodies it has an affinity with. The sandwich complex would consist of the analyte substance, capture and detection antibodies to the analyte substance, the redox active material and a capture surface or particle at an electrode. The capturing system may be an aptamer or nucleotide assay, or a selective absorption or other chemical binding interaction. The electrodes which form the Galvanic cell are an anode and cathode, and where the substance complex is the anode then a cathode redox active material may be coating the cathode electrode.

In some embodiments, the analyte capturing system may be based on a protein binding mechanism such as state of the art sandwich and bridge type immunoassays. For example, the analyte capturing system may employ a streptavidin to biotin capture mechanism. In those embodiments, the biotin or biotin coated inert particles, may be configured to be added to the streptavidin magnetic particles or streptavidin functionalised capture electrode to compete with the signal particle or to deliberately aggregate magnetic particles and capture the zinc within, for adjusting sensitivity or analytical concentration ranges.

The galvanic detection device may be a sandwich immunoassay where the target analyte is configured to be captured and sandwiched between two antibodies. The two different antibodies specific to an analyte may be configured to be attached to the anode or cathode active signal label and to the capture anode or cathode electrode or magnetic particle. This allows the signal label to be in contact with the detector electrode and measured.

The galvanic detection device may be an immunoassay test device. The galvanic detection device may be a detection device in a laboratory analyser. Without wishing to be bound by theory, the galvanic detection device may replace existing laboratory detection devices employing optical detection of assay complex sandwich constructs. The galvanic detection device may be a microfluidic test device. The galvanic detection device may be a screen printed test device. The screen printed test device may comprise one or more of: a flexible patterned polymer, an adhesive film incorporating screen printed conductive tracks, at least one pair of detection electrodes, each pair of detection electrodes comprising an anode and a cathode detection electrode for galvanic detection of the analyte (e.g. via assay detection label particles), and a control electrode for each pair of detection electrodes.

In some embodiments, the galvanic detection device may be a microfluidic assay test device comprising anode particle functionalised with a secondary antibody, a biotin functionalised with a primary antibody, and a magnetic particle functionalised with streptavidin. In use, when a sample containing an analyte is placed in the device an assay complex sandwich construct may be formed. The assay complex construct may have the following structure:

| | | | |
|---|---|---|---|
| anode particle- | analyte | primary antibody- | streptavidin-magnetic |
| secondary antibody | | biotin | particle |

The galvanic detection device may comprise a magnet to attract the complex having a magnetic particle and to deliver the complex to a detector electrode. The magnet may be static and it may be disposed behind the electrode to attract the complex comprising the analyte towards the electrode. The magnet may be disposed along the channel of the microfluidic device and be moved into place. Advantageously, this configuration allows additional manipulation and even allows a background measurement to be made at the detector just prior to detection of the analyte before the magnet moves and delivers the complex. This can provide a better correction of the background readings/noise and therefore improve the selectivity and sensitivity of the device.

A very important advantage to using the magnet is that there is a force pulling the complex onto the electrode. This increases the instances of detection particle making electrical contact with the electrode. It also allows close packing of complexes. Therefore, the greater the concentration of analyte in a sample, the more complexes are formed in the device, and therefore, the greater the signal that is detected. Close packing is important as detection particles can contact each other and form a pile in electrical connection.

Prior art detection methods based on direct measurement of magnetic particle delivery of silver detection particles with voltammetry have failed to accurately detect the analyte concentration in a sample due to insufficient contact with the detection electrode. Advantageously, the galvanic detection device (and methods employing said device) may avoid these problems and provide accurate determination of the concentration of an analyte in a sample.

In other (less preferred) embodiments, the galvanic detection device may be a microfluidic assay test device comprising anode particle functionalised with a secondary antibody, a biotin functionalised with a primary antibody, and a detection electrode having a surface functionalised with streptavidin. In use, when a sample containing an analyte is placed in the device an assay complex sandwich construct may be formed. The assay complex construct may have the following structure:

| | | | |
|---|---|---|---|
| anode particle- | analyte | primary antibody- | streptavidin-electrode |
| secondary antibody | | biotin | |

In this embodiment, the complex may not include a magnetic particle. The complex may be configured to be captured at the detector by diffusion or microfluidic flow manipulation. The detector electrode may comprise having the functionalised capture agent (e.g. streptavidin) coated on its surface.

The anode electrode and the cathode electrode may be connected to each other to the galvanic detection device by any suitable means. For example, the anode electrode and the cathode electrode may be connected to each other or to the galvanic detection device with an electric conductor, by screen printed carbon tracks. In some embodiments, the anode electrode and the cathode electrode are connected to the galvanic detection device by separate electrically connected screen printed carbon tracks.

The galvanic detection device may comprise at least one magnet disposed adjacent to a detection electrode. The magnet may define any suitable shape or form. The magnet may be narrower than a detection electrode. Advantageously, in use, a narrow magnet brings the front and back edges of the captured magnetic particles closer together to fit into measurement zone on electrode, reducing front and back edge magnetic particle gathering. The galvanic detection device may further comprise a magnet. The magnet may be positioned in close proximity to at least one of the detection anode and/or the detection cathode. The magnet may be stationary or it may be movable. The magnet may be positioned outside the galvanic detection device, but close to a channel of the galvanic detection device. In embodiments in which the galvanic detection device comprises magnetic particles (e.g. within a microfluidic channel of the device), the magnet may be configured to attract the magnetic particles. In embodiments in which the device is a microfluidic device, the detection electrodes may be disposed on a surface inside the channel of the microfluidic device. The magnet may be disposed outside the device, and behind the electrode (which is on a surface inside the channel). Advantageously, the magnetic particles are configured to be attracted to the magnet while also to the electrode thus making contact for a galvanic event between the magnetic particles and the electrode to occur. In embodiments in which the magnet is movable, if the magnet is moved, the particles will be attracted to the magnet and will therefore follow it. Advantageously, the magnet may be movable on and off the electrode to recharge the galvanic cell. The galvanic detection device may comprise an array of magnets. The array of magnets may comprise planar magnets laminated together in a Halbach type array where each magnets pole is adjacent to the opposing pole of the next magnet. Advantageously, this arrangement provides a more evenly distributed magnetic field across the detection electrode, also reducing strong magnet edge effects on magnetic particle distribution.

In embodiments in which the galvanic detection device comprises multiple pairs of detection electrodes, the galvanic detection device may comprise a functionalised electrode or a magnet in each of the pairs of detection electrodes. Alternatively, the galvanic detection device may comprise a magnet and/or a functionalised detection electrode in only one of the pairs of detection electrodes.

The galvanic detection device may be configured to be used with an electrolyte. The galvanic detection device may comprise an electrolyte. The electrolyte may contain a metal salt. The electrolyte may comprise any metal salt suitable for batteries and/or voltaic cells. Metal salts may comprise sulphates, chlorides, tetrafluoroborates, eutectics, organic salts, polymer-based salts, and the like. In some embodiments, the electrolyte comprises zinc chloride or other zinc salt, potassium chloride, and/or sodium chloride.

The electrodes may be positioned in a microfluidic channel. The microfluidic channel may be filled with an electrolyte. In use, the anode material substance complex may be captured on the anode electrode forming a spontaneous Galvanic cell when measured electrically.

The galvanic detection device may further comprise an electronic measuring device, such as an electrometer, a potentiometer, a galvanometer, an ammeter, a voltmeter (e.g. a high impedance voltmeter), and the like.

The galvanic detection device may comprise an assay detection label. The assay detection label may be a magnetic particle, or it may be attached to a magnetic particle, e.g. by physisorption, chemisorption, or a protein binding interaction. In use, the assay detection label may be configured to be captured by a detection electrode (the anode or cathode depending on the design) in the presence of a magnetic field. The magnetic field may be generated by a magnet. The magnet may be located in any suitable location. For example, the magnet may be disposed below, to a side, or above a test strip configured to receive a solution containing an analyte. The assay detection label may comprise a redox active agent, such as any redox active agent compatible with redox flow batteries. The assay detection label may comprise a metal, such as a metal capable to be used in anode and/or a cathode in a commercial battery. For example, the assay detection label may comprise one or more of: zinc, copper, nickel, cadmium, gold, silver, manganese, iron, aluminium, magnesium, and the like. In some embodiments, the assay detection label may comprise zinc, for example a zinc microparticle. The assay detection label may comprise a particle (e.g. a zinc particle) functionalised with an antibody. The assay detection label may be configured to form part of a sandwich assay complex, such as detection particle-antibody//analyte//antibody-biotin//streptavidin-magnetic particle, or detection particle-antibody//analyte//antibody-biotin//streptavidin-electrode. The assay detection label (or detection particle) may be configured to be captured at the capturing surface. In some embodiments, the capturing surface is present on a magnetic particle. The magnetic particle (comprising or functionalised with the capturing surface) may be configured to capture or otherwise bind to the assay detection label (or detection particle). The detection electrode may be configured to detect the presence of the assay detection label. The quantity of assay detection label (containing analyte) may be correlated to the quantity of analyte present in a sample.

In some embodiments, the galvanic detection device is a double antibody assay. The double antibody assay may comprise a detection label functionalised with an antibody adsorbed thereto. The antibody of the functionalised detection label may be configured to bind to one side of an analyte (in use). The other side of the analyte is configured to bind to a second antibody which is either absorbed to a magnetic particle present in the galvanic detection device, or absorbed on the surface of a detection electrode. The detection label (particle) may be configured to be brought to the detection electrode, e.g. under a magnetic field created by a magnet, or by diffusion and binding to the functionalised surface of the electrode.

The type and quantity of assay detection label may be optimised depending on the analyte concentration range to be measured and/or the sample type. For example, the size and number of zinc anode particles determine the galvanic charge range and can be optimised to suit a target analyte concentration range and sample type (e.g. blood, plasma, and transfer buffer solutions).

The galvanic detection device may further comprise an electrolyte. In use, the galvanic detection device may be configured to be in contact with (e.g. immersed in) an electrolyte. The electrolyte may contain substances where ions can flow and transport charge. The electrolyte may comprise a metal salt. In embodiments in which the detection label comprises a metal particle, the electrolyte may comprise a salt of the same metal as the metal particle. For example, in embodiments in which the assay detection label comprises zinc particles, the electrolyte may comprise a zinc salt, such as zinc chloride or zinc tetrafluoroborate. For assay detection labels with copper particles, the electrolyte may comprise a copper salt, such as copper sulphate. For assay detection labels with silver particles, the electrolyte may comprise a silver salt, such as silver acetate. The electrolyte may be aqueous or solvent based with ions. For example, the electrolyte may contain ions dissolved in an aqueous medium. The electrolyte may comprise a conducting polymer or a solid polymer electrolyte.

The electrolyte may be acidic or alkaline.

The metal salt in the electrolyte may be present in any suitable concentration, for example from about 0.1 to about 3 M, preferably from about 0.5M to about 1.5 M, or from about 1M to about 1.5 M, or from about 0.5M to about 1.2M, or of about 1M, or from about 1 M to about 2M, or from about 2M to about 3M.

The electrolyte may further comprise a potassium and/or a sodium salt (to increase conductivity). The electrolyte may further comprise a buffer. The buffer may control the pH for electrochemical reactions to take place in the device (acidic o alkaline). The buffer may also control the pH to be suitable for protein activity (e.g. antibody binding).

The electrolyte may comprise a surfactant. The surfactant may be present in a trace concentration, such as <5%, preferably 0.05% w/w to about 1% w/w. The surfactant may be any suitable surfactant, such as tween surfactant, Triton X-100, and the like.

The electrolyte may comprise zinc chloride. The concentration of zinc chloride may vary depending on the analyte for example in a concentration of from about 0.5 M to about 2 M, or about 0.75 M to about 1.5 M, or about 0.6 M to about 1 M.

The galvanic detection device may further comprise an electrical measurement circuit to allow current flow. The electrical measurement circuit may comprise an electrical conductor, such as a metallic (e.g. copper) cable, or printed carbon tracks.

In use, the detection device is configured to form a spontaneous Galvanic cell when the cathode and anode electrodes are connected to each other by a conductor and a measurement circuit and when redox active materials containing captured analyte from a sample are in contact with an electrolyte and delivered to their respective anode or cathode electrodes.

In some embodiments, and in use, when the galvanic detection device is in contact with the electrolyte, the magnetic and zinc particles complex is configured to be captured at the anode electrode having a magnet positioned in such a way that the magnetic particles are captured within the electrode area and pulled towards the anode electrode to cause intimate contact generating a galvanic cell and current flow.

The invention relates to a self-assembled cell whereby the redox active parts are delivered to their respective anode or cathode conductors during an assay process. The cell conductors circuit may be completed by the electrolyte and the measurement circuit, and current will flow after the redox active anode and cathode materials are in contact with their respective electrode conductor completing a Galvanic cell.

The amounts of anode or cathode redox active materials captured and reacted are related to the amount of substance/analyte assayed. Therefore, the electrical charge energy generated is also related to the amount of the substance/analyte present in a sample and can be measured by an electrometer or other electrical measurement instrument and data logging system.

The redox active anode or cathode material is designed to mix with the target substance/analyte and recognise and bind to it by a chemical or physical interaction such as immunological or size exclusion and capture, forming a complex which is captured on or moved to an electrode in the Galvanic cell. Once the cell is formed and current can flow electrical measurements such as voltage and current are recorded over time and used to determine the amount of the substance captured. The greater the amount of substance present the greater the amount of complexes are formed and the greater the electrical energy produced. This is a form of electrochemical measurement analysis, and may be termed ‘immunogalvanometry’ or ‘galvanometric analysis’. Other forms are voltammetry, coulometry, amperometry, which generally drive redox reactions by means of applying potential differences and driving current through the cell, and by doing so can be affected by interfering reactions. Potentiometry measures a potential difference between the sensing element and a reference electrode with minimal current flow. This invention discloses an assay method which harnesses a spontaneous electrochemical galvanic cell reaction. The assay mechanism and self-assembly completes the galvanic cell and creates the internal power source in the cell which is then measured with electrical instrumentation.

A galvanic cell has the advantage of reducing the effects of competing side reactions which occur commonly in electrochemical assays, for example in glucose in blood using amperometry. In these cases current is driven through the electrodes in the sample using an external power source and interfering reactions can freely occur and are commonplace.

Once the anode or cathode redox active materials have been captured at the respective electrode, they are spontaneously oxidised or reduced allowing galvanic current to be measured. Additionally, like a rechargeable battery the cell can be ‘recharged’ by applying a reducing potential to the anode. The loaded cell voltage can then again be measured and charge calculated and related to the substance's concentration for verification. This additional process may be useful for quality control and increasing sensitivity and specificity.

A construct where either the cathode or anode material is already deposited on its respective conductor while anode or cathode material is delivered to its respective conductor during the assay process may also be employed. For example, the cathode active material is deposited on its electrode during manufacture and the anode active material is captured on its electrode during the galvanic electrochemical ‘energy diagnostics’ assay.

The design is suited to laboratory analysers with automated liquid handling, and to point of care or home test instruments with microfluidic test strips comprising reagents dried down within, or hybrid wet and dry test strips, or with external liquid reagents for sample transfer.

In laboratory analysers the sample to be assayed is mixed with liquid reagents and flowed over the electrodes. A signal develops over the first galvanic electrode with the functionalized surface, or with a magnet behind the electrode where functionalized magnetic capture beads are used. The electrochemical sensor cell may be washed with further liquid reagent and electrochemically cleaned by passing a current through the sensor electrode and a third cleaning electrode. This permits multiple use for as long as the second galvanic electrode lasts. For example, employing the sensor electrode in anode format the cathode electrode may be used until it is completely reduced galvanically before replacement.

In microfluidic test strips the reagents may be dried down as in typical lateral flow tests where porous or fibrous absorbent materials are previously loaded with wet reagent and dried before being laminated into the front end of the strip. The test sample is applied to the front end where it is absorbed by the materials thus resuspending and engaging with the reagents. The sample reagent mixture flows through the microfluidic device and over the galvanic electrodes allowing the electrochemical measurements and analysis to be performed.

In hybrid wet and dry test strips the sample may be mixed with reagent in a separate sample tube to permit sample transfer, handling and separation of unwanted components before being applied to the test strip for electrochemical analysis.

Screen printing is a well-established process for laying down conductive tracks and electrodes for flexible electronics and sensor devices, e.g., ‘Screen-Printing Electrochemical Architectures’, 2016, ISBN: 978-3-319-25193-6, which is hereby incorporated by reference. Microfluidic test devices for electrochemical or optical detection methods are also well established. They are commonly produced by moulding plastics or laminating patterned polymer and adhesive films to define liquid channels, chambers and detection areas, e.g., ‘Biomedical Applications of Microfluidic Devices’, 2020, ISBN: 978-0-128-18791-3, which is hereby incorporated by reference.

The invention described herein is demonstrated on microfluidic test strips which have been produced using flexible patterned polymer and adhesive films incorporating screen printed conductive tracks and anode and cathode electrodes for galvanic detection of the assay label particles. The cathode electrode is a screen printed silver with silver chloride film and the anode electrode is a screen printed carbon film, both which are connected to the electronic measuring device by separate screen printed carbon tracks. The assay detection label is a zinc microparticle which is attached to a magnetic microparticle via physisorption, chemisorption or a protein binding interaction, and captured over the anode electrode in the presence of a magnetic field from a magnet positioned behind the anode in the microfluidic test strip. The magnet can be below, to a side or above the test strip, the later may be favourable for avoiding the influence of gravity on the assay.

The galvanic electrochemical reaction and standard reduction potentials can be described as:

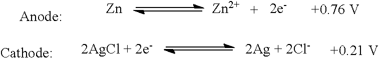

The galvanic redox couple therefore delivers a theoretical voltage of 0.97 V.

A galvanic current is spontaneously produced when the anode and cathode immersed in a suitable common electrolyte are electrically connected across their connector ends. The electrical measuring instrument is configured to connect the connector ends and measure the voltage and current over time. The electrical charge can be measured allowing the amount of zinc label on the anode to be calculated, this value can be used to measure the amount of target analyte in an assay. The size and number of zinc anode particles determine the galvanic charge range and can be optimised to suit a target analyte concentration range and sample type, for example blood, plasma, and transfer buffer solutions.

For more evenly distributing magnetic particles a suitably designed magnet is required. For example, a magnet narrower than the detection electrode brings the front and back edges of the captured magnetic particles closer together to fit into measurement zone on electrode, reducing front and back edge magnetic particle gathering. Magnet arrays can be used, for example planar magnets laminated together in a Halbach type array where each magnets pole is adjacent to the opposing pole of the next to provide a more evenly distributed magnetic field across the detection electrode, also reducing strong magnet edge effects on magnetic particle distribution.

Where there is no zinc bound to the magnetic particle there is a near zero current produced and as bound zinc captured on the magnetic particle increases the current increases. The magnetic particles do not themselves allow a Galvanic cell to form, however in a format where this is desired the particle may be both magnetic and redox active.

The electrode may be flat and smooth where the particle size combination permits the active anode or cathode material to contact the electrode. The electrode can be porous so the particles are pulled into micro domains providing greater surface area and opportunity for the redox active material to touch the electrode.

The electrolyte can be formulated a pH to suit both certain biological interactions and electrochemical reactions.

For streptavidin to biotin capture mechanisms the biotin or biotin coated inert particles, could be added to the streptavidin magnetic particles or streptavidin functionalised capture electrode to compete with the signal particle or to deliberately aggregate magnetic particles and capture the zinc within, for adjusting sensitivity or analytical concentration ranges. The relative and actual size of the magnetic and anode or cathode particles can be adjusted to suit sensitivity or analytical concentration ranges. For example, larger magnetic particles are drawn to the magnet electrode more strongly and faster than smaller magnetic particles. Agglomeration and aggregation of the particle complexes can be leveraged to enhance the detection and analytical specificity. Additional carrier particles can be incorporated to attach the electrochemical redox active cathode or anode particles where the carrier particles are also functionalised with chemistries and proteins and able to form additional complexes.

The assay format can adopt other protein binding mechanisms such as state of the art sandwich and bridge type immunoassays and other existing and new assay schemes. The solid phase for immobilising the detection antibody (ligand) may, for example, be the surface of the electrode, the surface of a microbead (optionally magnetically susceptible), the surface of a fluidic chamber, or the surface of a membrane.

The assay can be formatted as a sandwich immunoassay where the target analyte is captured and sandwiched between two antibodies. The two different antibodies specific to an analyte are attached to the anode or cathode active signal label and to the capture anode or cathode electrode or magnetic particle. This allows the signal label to be in contact with the detector electrode and measured as illustrated in the assay schemes, FIGS. 1(a)-(f).

The electrolyte formulation is also critical. An electrolyte that is suited to physiological biological biochemistries as well as enabling the formation of a galvanic cell is required. This can be performed in two step process where the biochemistry is first performed in a solution whereafter a second electrolyte component is added to support the galvanic reaction at the electrodes. The solution or buffer can be formulated to suit both sample biochemistry and galvanic cell electrochemistry, by formulating a buffer at a suitable pH and incorporating anode or cathode cations or anions according to the galvanic cell chemistry energetics requirement, for example in a pH range 4 to 9 and containing redox couple species enabling a Galvanic cell.

The anode and cathode electrodes and connector tracks may be printed onto flexible polymer film, and the microfluidic channels and chambers patterned in adhesive laminate with a cover film completing the test strip embodiment.

The galvanic current can be measured using commercially available sensitive voltmeters, ammeters or galvanometers. Simple circuits capable of sensing low current voltages and linked to data acquisition computer systems for logging data may be employed, and may be miniaturised into a portable custom electronic reader with user interface and measurement software.

Multiple capturing systems (e.g. particle systems) may be employed, for example, a preformed redox active particle coupled to a chemically or affinity functionalised particle making the label complex which is then captured by a detection particle or surface (e.g. be the surface of the electrode, the surface of a microbead (optionally magnetically susceptible), the surface of a fluidic chamber, or the surface of a membrane) in the presence of analyte, and forming a complex with the analyte.

The particles can be prepared in what is termed a reagent mixture which can be deposited into the test strip reagent allocated zones. The components of the reagent can be altogether in one mixture or in separate mixtures, and can be deposited in one zone or multiple zones. The reagents can be designed to assay a wide range of analyte, for example a first zone having reagents for a low concentration of analyte and second zone for a high concentration of analyte. The reagent zones could be in the same channel and exposed to the sample analyte in sequence. Assaying a low concentration of analyte would benefit from contact with the first zone and prevent issues of steric hindrance and kinetic inhibition in forming the detection complex, and be less impacted by a higher analyte optimised reagent even if mixed together subsequently in the second zone. High levels of analyte which would cause saturation and an assay hook effect in the first zone would benefit in the second zone which is optimised for the high level. Multiple reagent zones optimised for discreet analyte concentrations across a channel could be employed to extend the assay analyte concentration range. The zones would be exposed to analyte in sequence by fluidic control and manipulation in such a way that the sample volume is consistent.

For systems using magnetic capture particles, the relative and actual sizes of the anode or cathode particles to the magnetic particle size can be varied. For example, smaller than magnetic anode particles allow more anode particles to be captured per magnetic particle. The actual size of the magnetic particles and the magnet size and position will determine the magnetic force and effectiveness of capturing and delivering the anode particle to close contacting the electrode. The packing density of the particles will also be influenced by particle sizes, and can be formatted to pack in such a way that layers of contacting anode or cathode particles can be created to increase the Galvanic current range possible. The particles' size is also a major factor in the assay kinetics and binding and complexing rates with the analyte.

In a second aspect, the present disclosure relates to a method for the determination of an analyte or substance by means of spontaneous electrochemical reactions at anode and cathode electrodes in an electrolyte and arranged to form a measurable Galvanic cell.

A spontaneous redox reaction generates electromotive force and electrical energy which is measured and can be used in an assay to measure the amount of a target substance.

When an anode and a cathode made of redox active materials of differing electrochemical reduction potentials are both together in a suitable electrolyte, electrical current can flow if the dry conductive ends are connected by an electrical conductor or measuring instrument.

A method for determining an analyte substance in an electrochemical detection assay device, comprising:
(a) Forming an analyte substance complex by capturing the analyte substance and complexing it with a redox active material to assemble part of a measurable Galvanic cell, optionally wherein the capturing step is performed by affinity binding;
(b) allowing the analyte substance complex to travel in an electrolyte to a detection electrode and spontaneously capturing the analyte substance complex at the detection electrode to assemble part of a measurable Galvanic cell;
(c) electrically connecting the detection electrode (cathode/anode) to a complementary detection electrode (anode/cathode) to activate a measurable Galvanic cell;
(d) optionally connecting the control electrode to a complimentary control electrode within an electrolyte to an electrical measurement circuit to allow flow of any background Galvanic electrical current;
(e) providing a measurement circuit between the detection electrodes, optionally wherein the measurement circuit includes an operational amplifier with a load resistor;
(f) measuring voltage and current changes in the measurement circuit, optionally wherein this step is performed using a data acquisition and data processing reader device, further optionally wherein the data acquisition and data processing reader device is an analogue to digital data acquisition and data processing reader device.

The method may further comprise measuring a background reading with a pair of control electrodes in contact with the electrolyte.

In this method, the galvanic detector may link to the conductor track to the connector/instrument.

The anode/cathode detection electrodes and any control electrodes if present may be in contact with electrolyte. For example, in microfluidic test devices, the anode and cathode (and any control/reference electrodes) may be in contact with the test strip and may be wet by electrolyte. The dry part sealed under adhesive may be conductor tracks leading to exposed connectors ends for connection to a measurement instrument. Galvanic coupling may take place between the two detector electrodes anode and cathode. Where there are control electrodes present, the 2 controls may be separate background galvanic measurements, and they may be performed for quality control.

In another aspect, the present invention is directed to a method for capturing a substance or analyte and transferring it to an electrode and electrolyte to complete a Galvanic cell.

The step of capturing the analyte substance complex may be performed by any suitable means, such as by attracting the analyte substance complex to the electrode in a magnetic field, or by means of a chemical or electrochemical reaction.

The electrochemical detection assay device may be a microfluidic device.

The step of capturing the analyte substance and complexing it with a redox active material may comprise contacting a sample comprising an analyte with an electrolyte comprising a substance capable of capturing or binding to the analyte.

The method may comprise capturing multiple substances on detection electrodes to complete a Galvanic cell. In some embodiments, the method comprises capturing multiple substances on detection electrodes to complete multiple Galvanic cells. The step of “capturing” may be performed directly on the surface of a detection electrode, or indirectly on a capturing system (e.g. on a particle), which is then moved to a detection electrode (e.g. by fluid flow, electrochemical reaction, chemical reaction, or magnetic field).

In another aspect, there is provided a galvanometric method for the determination of an analyte or substance by means of spontaneous electrochemical reactions at anode and cathode electrodes in an electrolyte and arranged to form a measurable Galvanic cell, the method comprising:
(a) depositing a sample in a galvanometric detection device, wherein the galvanometric detection device comprises:
   (i) a detection particle having a redox active material and a binding element capable of binding to an analyte from the sample;
   (ii) a capturing surface capable of binding to the detection particle;
   (iv) (iii) at least a pair of detection electrodes optionally a pair of control electrodes;
(b) allowing an analyte in the sample to form a detection complex with the detection particle;
(c) optionally attracting the detection complex to a detection electrode;
(d) allowing the detection complex to bind to the capturing surface;
(e) placing the pair of detection electrodes, in contact with an electrolyte;
(f) optionally placing the pair of control electrodes in contact with an electrolyte;
(g) electrically connecting the pair of detection electrodes to a measuring circuit to activate a measurable Galvanic cell;
(h) optionally electrically connecting the pair of control electrodes to a measuring circuit to activate a measurable Galvanic cell background;
(i) measuring voltage and current changes in the spontaneous Galvanic cell;
(j) correlating the measured voltage and current changes to the concentration of the analyte in the sample.

The detection particle may comprise a metal particle, such as a zinc particle, biotin, and a moiety capable of binding to the analyte, optionally wherein the moiety capable of binding to the analyte is an antibody. The capturing surface may comprise streptavidin. The streptavidin may be capable of complexing with the biotin of the detection particle. In other embodiments, the detection particle may comprise streptavidin and the capturing surface may comprise biotin.

The capturing surface may be present on the detection electrode. For example, the surface of the detection electrode may be functionalised with the capturing agent (e.g. streptavidin). In those embodiments, the detection particle may be configured to move to the capturing surface of the detection electrode by fluid control.

The capturing surface may be present on a magnetic particle. For example, the galvanometric detection device may comprise one or more magnetic particles functionalised with the capturing agent (e.g. streptavidin). The galvanometric detection device may comprise a magnet. The magnet may be disposed near the detection electrode (e.g. above, below, or adjacent to the detection electrode), to attract the magnetic particle once it has complexed with the detection particle and the analyte towards the electrode in order to establish spontaneous electrochemical reaction and to determine the concentration of the analyte in the sample. The method may comprise moving the magnet towards the detection electrode to guide the complex comprising the magnetic particle and the analyte towards the detection electrode.

The detection methods of any of the aspects of the invention may be performed with the galvanic detection device of the invention.

For the avoidance of doubt, any aspect of the invention may be combined with any other aspect of the invention disclosed herein.

Within the context of this application, the invention may not include any steps involving the collection of a sample from a human or animal body.

The methods disclosed herein may further include a step of sample preparation, such as centrifugation, pre-treatment of a sample with an electrolyte or capturing system, or the like.

Furthermore, the invention comprises a method of assaying an analyte substance by means of capturing the substance and complexing it with redox active materials to complete a measurable Galvanic cell. The substance complex may be captured near or on an electrode in electrolyte to complete a measurable Galvanic cell.

The complex may be transferred to the electrode by fluidic control and flow. Multiple substances complexes transferred to multiple electrodes to form multiple cells could be employed to distinguish analytes by each complex forming different redox cell potentials.

The electrolyte may comprise ions capable of flowing and transporting charge. The electrolyte may be aqueous and solvent based with ions or a conducting polymer or a solid polymer electrolyte. The substance capturing surface may be functionalised electrical conductors and electrodes which bind the analyte substance.

The capturing system may be an affinity protein or antibody, or be a functionalised particle which such. The capturing system may comprise a size exclusion filter with electrical conductors and electrodes forming a measurable Galvanic circuit. The capturing particle may be a magnetically susceptible particle which can be transferred to the electrode by a magnetic field or magnet behind the electrode, or be susceptible to other self-assembly means.

The analyte capturing system may be an immunoassay, for example a sandwich type immunoassay where the analyte is held between the capture surface and the substance complex by antibodies it has an affinity with. The sandwich complex would consist of the analyte substance, capture and detection antibodies to the analyte substance, the redox active material and a capture surface or particle at an electrode. The capturing system may be an aptamer or nucleotide assay, or a selective absorption or other chemical binding interaction. The electrodes which form the Galvanic cell are an anode and cathode, and where the substance complex is the anode then a cathode redox active material is coating the cathode electrode. The electrodes may be positioned in a microfluidic channel filled with electrolyte and the anode material substance complex is captured on the anode electrode forming a spontaneous Galvanic cell when measured electrically. The redox active material in the analyte substance complex should be close enough to the electrode to allow electron transfer. An example of Galvanic cell is where the cathode is a silver and silver chloride blend deposited on the cathode electrode, and the anode material is a zinc nano or micro particle, and the electrolyte contains zinc chloride or other zinc salt. Silver redox photochemistry may form the redox chemistry by using silver particles in the analyte substance complex. Silver particles captured on the cathode electrode collector and then exposed to light and are oxidised forming a redox active chemistry couple with an anode chemistry already in place to complete the Galvanic cell. The redox active materials can be any combination which in theory and practice form a Galvanic cell. The reagent may be deposited in the test strip and become resuspended or dissolved in the sample, or be within an analyte extractor buffer tube or its associated filter nozzle or sampler, and be applied to the strip already in suspension. The reagent may be immobilised in a material positioned at or near the strip entry or in the strip channel. The reagent may be deposited in the strip so the analyte sample volume meets sequential reagent zones in a channel where each zone is optimised for an analyte concentration range.

### BRIEF DESCRIPTION OF DRAWINGS

FIG. 1a illustrates a system where a galvanic cell is formed by the self assembly of sandwich type immunoassay reagents via capture directly onto an electrode including analyte.

FIG. 1b illustrates a system where a galvanic cell is formed by the self assembly of sandwich type immunoassay reagents via magnetic particle capture onto an electrode including analyte.

FIG. 1c illustrates a system where a galvanic cell is formed by the self assembly of anode-biotin particle to streptavidin-magnetic particle capture onto an electrode where the magnetic particle is smaller than the anode particle.

FIG. 1d illustrates a system where a galvanic cell is formed by the self assembly of anode-biotin particle to streptavidin-magnetic particle capture onto an electrode where the magnetic particle is larger than the anode particle.

FIG. 1e illustrates a system where a galvanic cell is formed by the self assembly of anode particle absorbed onto magnetic particle capture onto an electrode where the magnetic particle is smaller than the anode particle.

FIG. 1f illustrates a system where a galvanic cell is formed by the self assembly of anode particle absorbed onto magnetic particle capture onto an electrode where the magnetic particle is larger than the anode particle.

FIG. 2a shows the measurement system comprising the microfluidic device, measurement circuit and data acquisition device (connected to PC not shown).

FIG. 2b shows the microfluidic device, its electrodes, connectors and magnet.

FIG. 2c shows the microfluidic devices electrodes after a test where the anode particles absorbed to the magnetic particles have been captured at the anode electrode and are clearly visible.

FIG. 3a shows a plot of voltage against time measurement response for increasing amounts of captured anode particles over six separate device test runs.

FIG. 3b shows a plot of Galvanic cell charge against relative number of captured anode particles based on the test data shown in FIG. 3a.

### DETAILED DESCRIPTION

The galvanic detection devices and methods of the invention have been demonstrated herein in strip tests. The galvanic detection device may be a microfluidic device comprising screen printed electrodes, such as those employed in glucose biosensors.

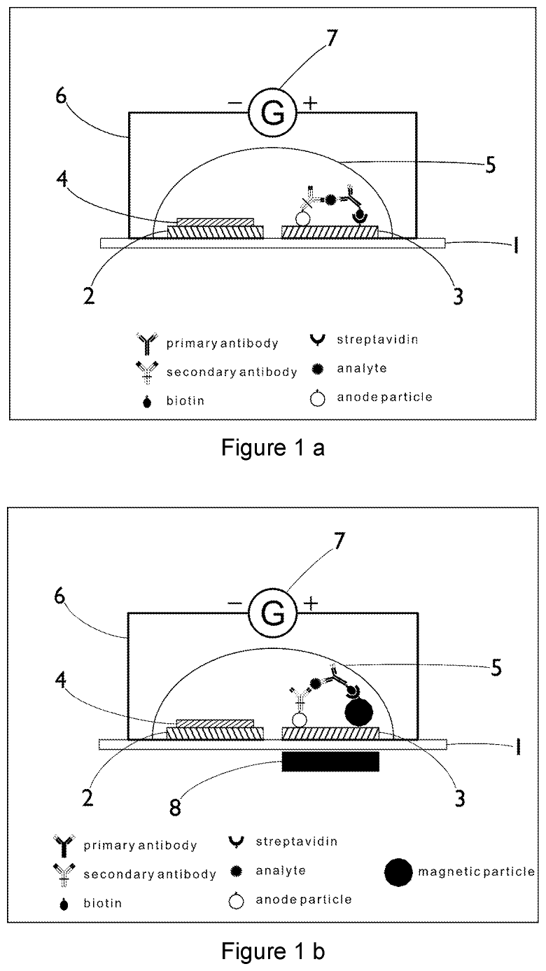

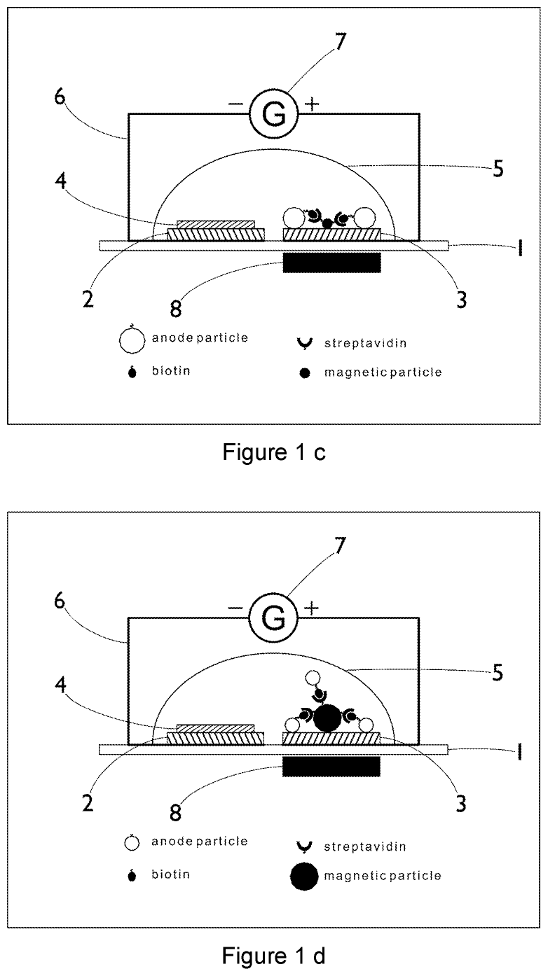

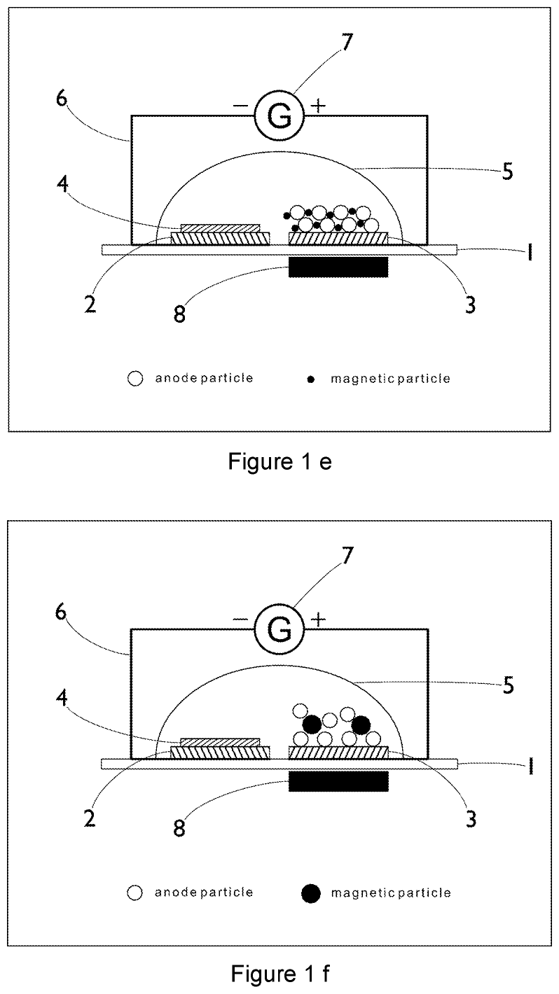

In the exemplary embodiment illustrated herein, the galvanic detection device is a double antibody assay. The double antibody assay comprises a detection label functionalised with an antibody adsorbed thereto. The antibody of the functionalised detection label may be configured to bind to one side of an analyte (in use). The other side of the analyte is configured to bind to a second antibody which is either absorbed to a magnetic particle present in the galvanic detection device, or absorbed on the surface of a detection electrode. The detection label (particle) may be configured to be brought to the detection electrode, e.g. under a magnetic field created by a magnet (FIG. 1b), or by diffusion and binding to the functionalised surface of the electrode (FIG. 1a).

The invention described herein is demonstrated on microfluidic test strips which have been produced using flexible patterned polymer and adhesive films incorporating screen printed conductive tracks and anode and cathode electrodes for galvanic detection of the assay label particles. The cathode electrode is a screen printed silver with silver chloride film and the anode electrode is a screen printed carbon film, both which are connected to the electronic measuring device by separate screen printed carbon tracks. The assay detection label is a zinc microparticle which is attached to a magnetic microparticle via physisorption, chemisorption or a protein binding interaction, and captured over the anode electrode in the presence of a magnetic field from a magnet positioned behind the anode in the microfluidic test strip. The magnet can be below, to a side or above the test strip, the later may be favourable for avoiding the influence of gravity on the assay.

The galvanic electrochemical reaction and standard reduction potentials can be described as:

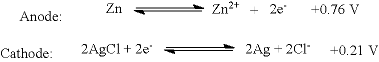

The galvanic redox couple therefore delivers a theoretical voltage of 0.97 V.

A galvanic current is spontaneously produced when the anode and cathode immersed in a suitable common electrolyte are electrically connected across their connector ends. The electrical measuring instrument is configured to connect the connector ends and measure the voltage and current over time. The electrical charge can be measured allowing the amount of zinc label on the anode to be calculated, this value can be used to measure the amount of target analyte in an assay. The size and number of zinc anode particles determine the galvanic charge range and can be optimised to suit a target analyte concentration range and sample type, for example blood, plasma, and transfer buffer solutions.

In a first embodiment (FIG. 1a), the galvanic detection device is a microfluidic assay test device comprising anode particle functionalised with a secondary antibody, a biotin functionalised with a primary antibody, and a detection electrode having a surface functionalised with streptavidin. In use, when a sample containing an analyte is placed in the device an assay complex sandwich construct may be formed. The assay complex construct may have the following structure:

| | | | |
|---|---|---|---|
| anode particle (zinc | analyte | primary antibody- | streptavidin-electrode |
| particle)-secondary | | biotin | |
| antibody | | | |

In this embodiment, the complex may not include a magnetic particle. The complex may be configured to be captured at the detector by diffusion or microfluidic flow manipulation. The detector electrode may comprise have the functionalised capture agent (e.g. streptavidin) coated on its surface.

With reference to FIG. 1a, a substrate 1 with two electrodes, a cathode 2 and an anode 3, are covered with an electrolyte 5 and connected 6 externally and electrically by a galvanometer 7. The cathode electrode is covered with a redox active material 4. The anode is initially uncoated, and no current flows though the galvanometer. The analyte antibody biotin complex sandwich comprises redox active material (e.g. a metal or metal ion). In the presence of analyte, the complex assembles and the detection complex is delivered close to the anode and making contact. In order for the redox active material to be detected at the anode, good contact between the redox active material of the detection particle and the anode is required. Appropriate electric contact of the redox active material in the detection complex with the anode is achieved when the detection complex is captured on the anode via immobilised streptavidin (i.e. the capturing surface is present on the anode). Since the cathode chemistry is at a higher standard reduction half-cell potential than the anode particle chemistry, current flows spontaneously and is measured at the galvanometer. The current measured is proportional to the amount of analyte present. FIG. 1b shows the system format for cell assembly using a magnet 8 and magnetic particles. The streptavidin is immobilised on the magnetic particles and drawn to the anode by the magnet situated behind the anode electrode, delivering an antibody analyte sandwich complex with anode particle close to the anode and making contact, completing the galvanic cell.

In some other embodiments (Shown in FIGS. 1b to 1f), the galvanic detection device is a microfluidic assay test device comprising anode particle functionalised with a secondary antibody, a biotin functionalised with a primary antibody, and a magnetic particle functionalised with streptavidin. In use, when a sample containing an analyte is placed in the device an assay complex sandwich construct is formed. The assay complex construct may have the following structure:

| | | | |
|---|---|---|---|
| anode particle- | analyte | primary antibody- | streptavidin-magnetic |
| secondary antibody | | biotin | particle |

The galvanic detection device comprises a magnet to attract the complex having a magnetic particle and to deliver the complex to a detector electrode. The magnet may be static and it may be disposed behind the electrode to attract attracting the complex comprising the analyte towards the electrode. Alternatively, the magnet may be movable and start at the strip of the microfluidic galvanic detection device and it may be moved close (adjacent to) the anode or cathode detection electrode for the detection electrode to enter in contact with the assay complex once it is formed. The magnet may be disposed along the channel of the microfluidic device and be moved into place. Advantageously, this configuration allows additional manipulation and even allows a background measurement to be made at the detector just prior to detection of the analyte before the magnet moves and delivers the complex. This can provide a better correction of the background readings/noise and therefore improve the selectivity and sensitivity of the device.

This invention was demonstrated using commercially available streptavidin magnetic particles and zinc particles, where the magnetic particles were mixed with zinc particles in a molar concentration of aqueous potassium chloride. An electrolyte mixture of aqueous 1 molar potassium chloride, about 0.5% tween surfactant and 0.75 molar zinc chloride was then added to the particles and mixed before being applied to the microfluidic strip. As the mixture flowed through the microfluidic channel and over the anode electrode and silver/silver chloride cathode, the magnetic and zinc particles complex to form a complex construct, which is captured at the anode electrode having a magnet positioned above in such a way that the magnetic particles are captured within the electrode area and pulled towards the anode electrode to cause intimate contact generating a galvanic cell and current flow. Unbound zinc particles flow past the electrode having the magnet behind and are not attracted to it thereby do not interfere. The galvanic current can flow while the microfluidic channel cell is full with electrolyte and also when emptied since the walls of the channel cell are sufficiently wet with electrolyte for the galvanic current to continue. Emptying the cell also removes potential interferences in the sample and unbound reagents.

For more evenly distributing magnetic particles a suitably designed magnet is employed. For example, a magnet narrower than the detection electrode brings the front and back edges of the captured magnetic particles closer together to fit into measurement zone on electrode, reducing front and back edge magnetic particle gathering. Magnet arrays can be used, for example planar magnets laminated together in a Halbach type array where each magnets pole is adjacent to the opposing pole of the next to provide a more evenly distributed magnetic field across the detection electrode, also reducing strong magnet edge effects on magnetic particle distribution.

Where there is no zinc bound to the magnetic particle there is a near zero current produced and as bound zinc captured on the magnetic particle increases the current increases. The magnetic particles do not themselves allow a Galvanic cell to form, however in a format where this is desired the particle may be both magnetic and redox active.

FIG. 1c shows a biotin coated anode particle bound to a streptavidin coated magnetic particle, where the magnetic particle is smaller than the anode particle. The attraction of the complex to the magnet and therefore electrode is weaker while the surface area of the anode particle contacting the anode electrode is greater. FIG. 1d shows a biotin coated anode particle bound to a streptavidin coated magnetic particle, where the magnetic particle is larger than the detection particle. The attraction of the complex to the magnet is stronger while the surface area of the anode particle contacting the anode electrode is smaller. This system can be used to characterise the assay components in development, to select the best actual and relative particle sizes and amounts for different analyte assays.

FIGS. 1e and 1f show anode particles bound directly to the magnetic particles by physisorption agglomeration or aggregation, and attracted to the magnet electrode. FIG. 1e shows anode particles bound to smaller magnetic particles and FIG. 1f shows anode particles bound to larger magnetic particles.

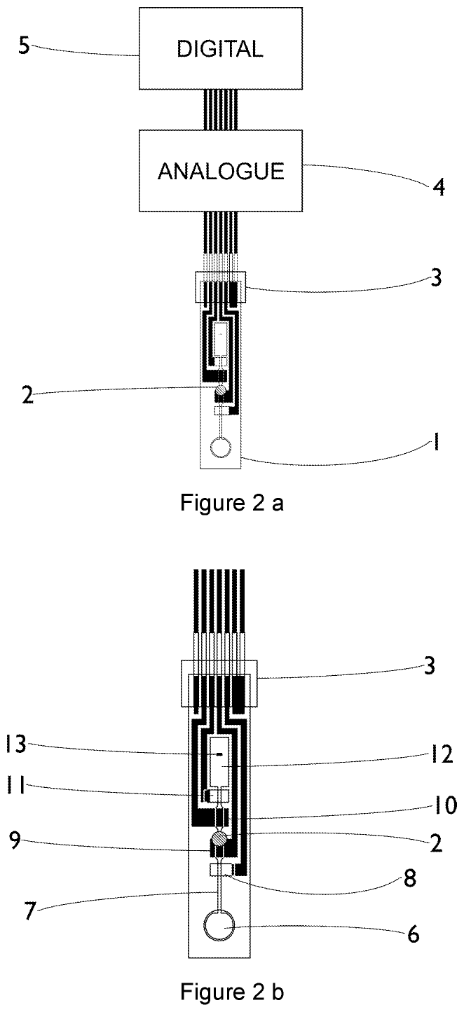

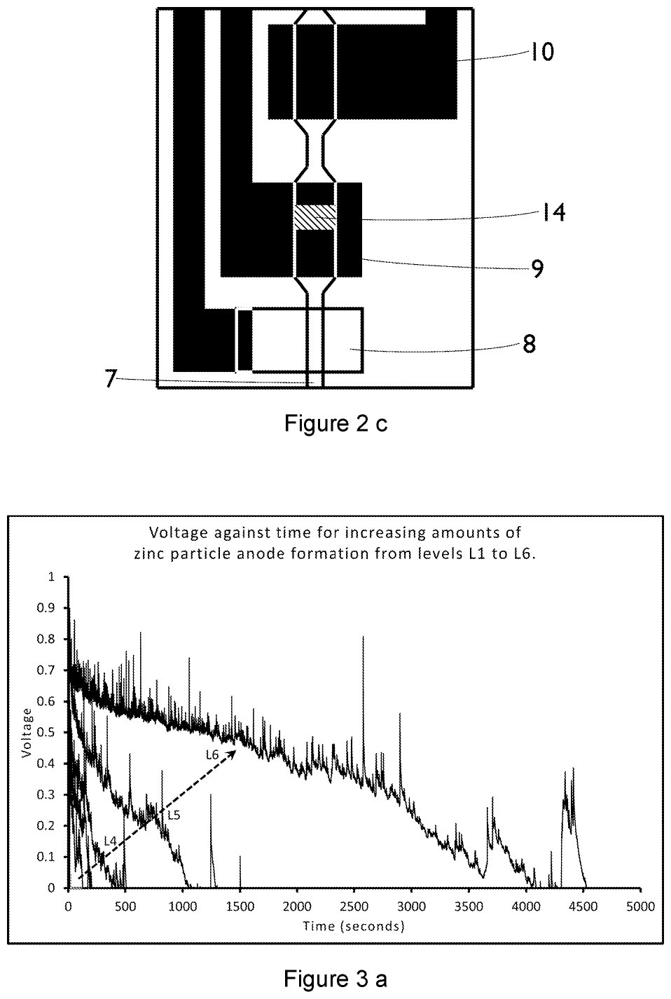

FIG. 2a shows the measurement system comprising the microfluidic device 1, magnet 2, the device connector 3, measurement circuit with operational amplifiers 4, and data acquisition device 5 (connected to PC not shown). Microfluidic test devices according to the invention can comprise electrodes and connector tracks printed onto a flexible polymer film such as PET, a double sided adhesive laminated over the electrodes and having pre-cut channels for the sample, covered then and laminated by a flexible polymer film confining the sample in the channel and a small vent is at the end of the channel so when the sample fills air is displaced. This test strip shown in FIG. 2, has two pairs of screen printed carbon tracks and anode electrodes, and a silver/silver chloride cathode over one pair of cathode electrodes. The device of FIG. 2 comprises a magnet is behind one anode. The strip is connected electrically via a standard edge connector.

FIG. 2b shows the microfluidic device sample inlet 6, its fluidic channel 7, its detection cathode electrode 8, and its detection anode electrode 9. The device also contains control electrodes for measuring the sample background and correct against any interferences, with control anode electrode 10, and control cathode electrode 11. The microfluidic device also contains a sink 12 for excess sample and to fluidic drive the sample through the channel by capillary or active forces with a vent 13 to release air or allow the air pressure inside the device to be varied.

FIG. 2c shows the microfluidic devices electrodes after a test where the anode particles absorbed to the magnetic particles have been captured at the anode electrode and are clearly visible 14. A measuring circuit which includes an operational amplifier with a load resistor was employed. Data was collected by measuring voltage and current changes in the measurement circuit using an analogue to digital data acquisition and data processing reader device

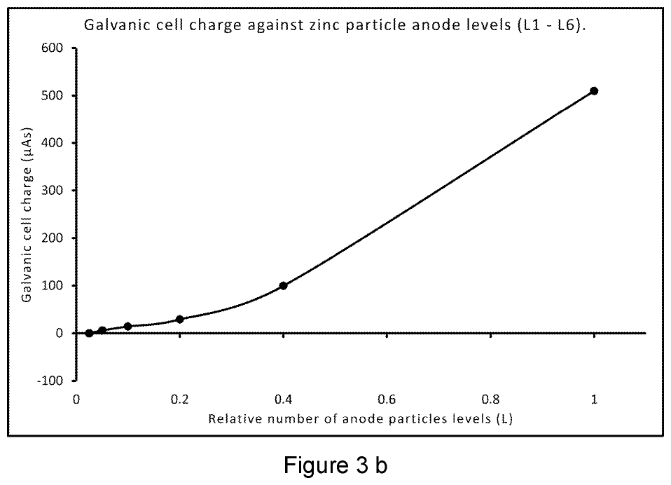

FIG. 3a shows a plot of voltage against time measurement response for increasing amounts of magnetically captured anode particles over six separate device test runs. The magnitude and duration of the voltage is proportional to the amount of zinc anode particles.

FIG. 3b shows a plot of Galvanic cell charge against relative number of captured anode particles based on the test data shown in FIG. 3a, and shows a correlation of charge with the amount of zinc anode particles captured at the anode electrode.

1. A galvanic detection device comprising at least one pair of detection electrodes, wherein at least one of:
a) at least one detection electrode comprises a capturing surface capable of capturing an analyte and/or particle; and/or
b) the galvanic detection device comprises a capturing surface system configured to travel to actively deliver an analyte and/or particle to at least one detection electrode.

2. The galvanic detection device of claim 1,
wherein the device comprises multiple pairs of detection electrodes, optionally wherein the galvanic detection device comprises two pairs of detection electrodes.

3. The galvanic detection device of claim 1, wherein the pair of detection electrodes comprises a non-reactive conductor electrode and a redox reactive electrode incorporating a redox active substance,
optionally wherein the non-reactive conductor electrode is configured to receive a redox active substance during an assay to form a galvanic cell, and
optionally wherein the redox active substance is be selected from the group consisting of zinc, silver, nickel, aluminium, copper, manganese, and salts and oxides thereof.

4. The galvanic detection device of claim 1,
wherein the capturing surface or capturing system is present on one or more of: an electrical conductor, an electrode, and/or a particle.

5. The galvanic detection device of claim 1,
wherein the capturing surface or capturing system comprises a capturing agent comprising one or more of: a size exclusion filter, an antibody, a protein, a protein binding system, an aptamer a nucleotide, a redox active material.

6. The galvanic detection device of claim 1,
wherein the capturing surface or capturing system is stationary or wherein the capturing surface or capturing system is configured to move towards a detection electrode.

7-8. (canceled)

9. The galvanic detection device of claim 1, wherein the device comprises;
a detection particle functionalised with an antibody for capturing an analyte and forming a detection complex comprising the detection particle and the analyte; and
a capturing surface for capturing the detection complex,
wherein the capturing system is configured to move towards a detection electrode by fluid flow, electrochemical reaction, chemical reaction, or under the influence of a magnetic field, and
wherein the detection particle is a magnetic particle and the galvanic detection device further comprises one or more magnets for attracting the magnetic detection particle towards or away from a detection electrode capturing surface.

10. The galvanic detection device of claim 1, wherein the capturing surface comprises a second antibody, wherein the second antibody is streptavidin or biotin.

11-14. (canceled)

15. The galvanic detection device of claim 1, wherein the particle is a magnetic particle and the galvanic detection device further comprises a magnet or magnetic field for attracting the magnetic particle.

16. The galvanic detection device of claim 1, wherein the detection device is a microfluidic test device capable of receiving a liquid sample for analysis.

17. The galvanic detection device of claim 1, wherein the detection device further comprises at least one electrolyte reagent.

18. The galvanic detection device of claim 1, further comprising an electronic measuring device.

19. (canceled)

20. A method for determining a sample's analyte substance in an electrolyte within an electrochemical detection assay microfluidic device, comprising:
(a) forming an analyte substance complex by capturing the analyte substance and complexing it with a redox active material to assemble part of a measurable Galvanic cell;
(b) allowing the analyte substance complex to travel in an electrolyte to a detection electrode and spontaneously capturing the analyte substance complex at the detection electrode to assemble part of a measurable Galvanic cell;
(c) electrically connecting the detection electrode (cathode/anode) to a complementary redox active detection electrode (anode/cathode) to activate a measurable Galvanic cell thereby providing a measurement circuit between the detection electrodes;
(d) optionally, connecting the control electrode to a complimentary control electrode within an electrolyte to an electrical measurement circuit to allow flow of any background Galvanic electrical current;
(e) providing a measurement circuit between the detection electrodes;
(f) measuring voltage and current changes in the measurement circuit while the microfluidic device is undergoing filling or undergoing emptying.

21. A galvanometric method for the determination of an analyte or substance in a sample by means of spontaneous electrochemical reactions at anode and cathode electrodes in an electrolyte and arranged to form a measurable Galvanic cell, the method comprising:
(a) depositing the sample in a galvanometric detection device, wherein the galvanometric detection device comprises:
(i) a detection particle having a redox active material and a binding element capable of binding to an analyte from the sample and forming a detection complex;
(ii) a capturing surface capable of binding the detection complex;
(iii) at least a pair of complementary detection electrodes wherein one electrode is redox active and one electrode receives the redox active material; and
(iv) optionally, a pair of complementary control anode and cathode electrodes;
(b) allowing an analyte in the sample to form a detection complex with the detection particle;
(c) allowing the detection complex to bind to the capturing surface of a detection electrode;
(d) optionally, attracting the detection complex to a detection electrode;
(e) enabling the pair of detection electrodes to be in contact with an electrolyte;
(f) optionally, enabling the pair of control electrodes to be in contact with an electrolyte;
(g) electrically connecting the pair of detection electrodes to a measuring circuit to activate a measurable Galvanic cell;
(h) optionally, electrically connecting the pair of control electrodes to a measuring circuit to activate a measurable Galvanic cell background;
(i) measuring voltage and current changes in the spontaneous Galvanic cell by an electrical circuit while the cell is undergoing filling or undergoing emptying; and
(j) correlating the measured voltage and current changes to the concentration of the analyte or substance in the sample.

22. The method of claim 21, wherein the detection particle comprises a metal particle.

23. The method of claim 21, wherein the capturing surface comprises streptavidin and the detection complex comprises biotin and wherein the streptavidin is capable of complexing with the biotin of the detection complex.

24. The method of claim 21, wherein the capturing surface is present on the detection electrode.

25. The method of claim 22, wherein the capturing surface is present on a magnetic particle, and wherein the galvanometric detection device comprises a magnet or magnetic field to attract the magnetic particle once the magnetic particle has complexed with the detection particle and the analyte towards the detection electrode in order to establish a spontaneous electrochemical reaction.

26. The method of claim 25, wherein the method comprises moving the magnet towards the detection electrode to guide the complex comprising the magnetic particle and the analyte towards the detection electrode.

27. The method of claim 20, wherein one of the electrodes of the at least one pair of complementary detection electrodes initially is inactive and does not contain redox active material, and wherein the galvanic cell is activated when the detection complex contacts the inactive detection electrode.

28. (canceled)
